(19) United States
(12) Patent Application Publication
(10) Pub. No.: US 2019/0343764 A1
(43) Pub. Date: Nov. 14, 2019

(54) EXCIPIENT BASE FORMULA, CHEWABLE FORMULA CONTAINING THE SAME, AND THE METHOD OF MAKING CHEWABLE TABLET

(71) Applicant: Nutraceutical Corporation, Park City, UT (US)

(72) Inventors: Max Willis, Park City, UT (US); Dave Bunch, Park City, UT (US); Scott Smith, Park City, UT (US); Rachel Kilroy, Park City, UT (US); Luke Wayment, Park City, UT (US); Lance Chau, Park City, UT (US); Ji Li, Park City, UT (US)

(21) Appl. No.: 15/979,161

(22) Filed: May 14, 2018

(51) Int. Cl. A61K 9/00; A61K 9/20; A61K 47/02; A61K 47/12; A61K 47/22; A61K 47/26; A61K 47/42; A61K 47/44

(52) U.S. Cl. CPC A61K 9/0056; A61K 9/2018; A61K 9/2068; A61K 9/2009; A61K 9/2095; A61K 31/07; A61K 47/12; A61K 47/22; A61K 47/26; A61K 47/42; A61K 47/44; A61K 47/02

(57) ABSTRACT

An excipient base formula for chewable tablet comprises from about 15% to about 55% by weight of xylitol; from about 20% to about 45% by weight of inulin; from about 1% to about 4% by weight of flowing agent; and from about 1% to about 4% by weight of lubricant based on the total weight of the excipient base formula. A chewable formula comprises at least one active ingredient and said excipient base formula, wherein inulin is not considered as the active ingredient. The chewable formula is suitable for a direct compaction to produce chewable tablet.

### FIELD OF THE DISCLOSURE

Embodiments of the present disclosure relate to the fields of dietary supplement, specialty pharmaceuticals, medical care and food, in particular to excipient base formula and chewable formula suitable for the production of chewable tablets.

### BACKGROUND OF THE DISCLOSURE

Chewable tablets are one type of solid dosage form, which break apart through chewing before ingestion. Chewable tablets serve as a good substitute for capsules and regular tablets, particularly for those who have difficulty in swallowing or where no water is available. Compared with other solid dosage forms (e.g., capsule and regular tablet), chewable tablets have a better active bioavailability by circumventing the need for disintegration. In addition, due to their advantageous palatable oral administration, portability, and ease of delivery, chewable tablets are marketable to a wide age range of consumers.

Chewable tablets may be suitable for direct compaction using various punch sizes ranging from ¼″ flat round shape to ⅞″ flat round shape, and various exotic punch shapes such as animal shapes and round dimple shapes. Compared with regular tablets, chewable tablets are not restricted to additional processes (e.g., dry/wet granulation), depending on different formulas. The simple one-step process renders chewable tablet a manufacture-friendly dosage form for developing dietary supplements, food, and pharmaceutical products.

Chewable tablets are typically composed of active ingredient(s), binder(s), lubricant(s) (e.g., magnesium stearate), flowing agent(s) (e.g., silica), sweetener(s), and flavor(s). The traditional chewable tablet composition enjoys a certain level of product advantages; however, the continuous improvement for its base formula is expected to meet the higher demand of manufacture, sensory evaluation and shelf life. For instance, magnesium stearate, which is a common lubricant in chewable tablets, is sensitive to blending time. Over-blending or over-use of a chewable formula with magnesium stearate can cause compression issues during manufacturing. Sorbitol is commonly used as a binder and sweetener for chewable tablets. However, sorbitol can cause sticking and low friability issues with exotic punch size during manufacture, as well as tablet sticking/spotting issues during storage. Moreover, the sticking issue of chewable tablets shortens the shelf life of chewable tablets, affecting the length of time that chewable tablets could be placed for sale on the shelf. The challenges in manufacture, sensory, and shelf life retard or slow the commercialization of chewable tablet products. To address these concerns, extra resources (time, personnel and equipment usage) are needed, resulting in a reduced economic supply chain of product development, manufacture, and selling. Under such circumstance, there is still a need for a novel, robust chewable tablet base formula that allows for the ease in manufacturing, a clean organoleptic experience, and an increase in product shelf life.

### BRIEF DESCRIPTION OF THE FIGURES

FIG. 1 shows the Attenuated Total Reflectance (ATR)—Fourier Transform Infrared (FTIR) spectra of the control chewable tablets of Formula A (i.e., at time=0), the 12-week chewable tablets of Formula A (i.e., at time=12 weeks under the accelerated stability test), granular xylitol, tart cherry, and organic inulin.

FIGS. 2A-2B shows the panel-based sensory taste test results of the magnesium chewable tablet samples of Formulas D, E and F. FIG. 2A shows the sensory attribute scores of chewable tablet samples of Formulas D, E, and F. FIG. 2B shows the sensory spider plots of chewable tablet samples of Formulas D, E and F.

### DETAILED DESCRIPTION OF THE DISCLOSURE

Embodiments of the present disclosure relate to a chewable formula for the production of chewable tablets that is manufacture-friendly, with a clear organoleptic experience, and has an extended shelf life. The chewable formula may be directly compressible into tablet form.

Embodiments of the present disclosure also relate to an excipient base formula for the preparation of said chewable formula. The disclosed excipient base formula enables the tablet to be directly compressible, free flowing and non-tacky, thus avoiding additional processes (e.g., dry/wet granulation) that can lead to the degradation of active ingredient(s) in the product and the increase in cost of manufacturing. Moreover, the disclosed excipient base formula can provide a clear organoleptic experience of relevant chewable tablets, and allow for an increase in the shelf life of chewable tablets.

The excipient base formula may comprise xylitol, inulin, silica, various lubricants, and various flavors. The excipient base formula offers a broad compositional percentage range for each ingredient, especially xylitol and inulin, depending on demands of different formulas. This gives a large flexibility for chewable tablet formulation. In one embodiment, the excipient base formula is composed of from about 15% to about 55% by weight of xylitol, from about 20% to about 45% by weight of inulin, from about 1% to about 4% by weight of flowing agent (e.g., silica), and from about 1% to about 4% by weight of lubricant(s), based on the total weight of the excipient base formula. The excipient base formula may optionally further include various accessory ingredients that are commonly known in the field. The accessory ingredients may include, but not limit to edible acid, color agent, surfactant, preservative, gum, chelating agent, antimicrobial agent, and etc. The weight percentage of accessory ingredients varies based on different chewable tablet formulas. As a non-limiting example, the excipient base formula may further comprise from about 3% to about 10% by weight of a flavoring agent based on the total weight of the excipient base formula.

Based on the excipient base formula, various fruit extract chewable tablets are developed and formulated. In further embodiments, the fruit extract chewable formula is composed of at least one fruit extract and an excipient base formula, wherein the excipient base formula comprises from about 15% to about 40% by weight of xylitol, from about 20% to about 40% by weight of inulin, from about 1% to about 3% by weight of flowing agent (e.g., silica), from about 1% to about 4% by weight of lubricant, and from 3% to 6% by weight of flavoring agent based on the total weight of the excipient base formula.

The term “excipient” as used herein refers to an inactive that serves as a vehicle or medium for an active ingredient to produce a formula suitable for the production of chewable tablets. Excipients can be small molecular sugar alcohols (e.g., sorbitol) or biopolymer materials (e.g., cellulose). Excipients may be included in the chewable formula for various purposes, such as long-term stabilization or bulking up (e.g., “bulking agents”, “fillers”, or “diluents”). Excipients are useful in the chewable tablet manufacturing process, such as to provide compressibility, increased lubricity or reduced friability during direct compaction.

The term “excipient base formula” as used herein refers to a base formula wherein none of the ingredients therein is an active ingredient.

The term “active ingredient” as used herein refers to a biologically or chemically active substance that provides nutritional or pharmaceutical value to the chewable tablet through oral administration.

Inulin

Inulin is a biopolymer composed of fructofuranose with a varying degree of polymerization (DP), ranging from 2 to 60 monomeric fructose units, linked to a terminal glucose molecule. In one embodiment of present disclosure, inulin has a DP of from about 2 to about 20, and preferably a DP of less than 10. The number-average molecular weight (Mn) of inulin used herein is about 5000 Da. The inulin material suitable for the present disclosure may be obtained from a variety of plants, such as Jerusalem artichoke tubers, dahlia tubers, or chicory roots.

Traditionally, inulin is used as a sort of prebiotic that stimulates the growth and/or activities of one or a limited number of microbial species in the gut microbiota that confers health benefits to the host, modulates the body immune system, and serves as a mild sweetener and stabilizer for food products. Inulin is commonly recommended to diabetics, since it is not absorbed and has no influence on blood glucose levels.

In the present application, inulin is predominantly used as an excipient rather than an active ingredient in the chewable tablet, since any nutritional or pharmaceutical value it possesses is not the purpose for which the chewable tablet is administered. Instead, inulin is used as an excipient and one of the components in the base formula of present application, since it functions as a binder for direct compaction, a filler, and/or a light sweetener. It has been found that when used as a filler and/or binder for regular tablets, inulin with appropriate degree of polymerization (DP), moisture content, and particle size offers good tableting properties and can significantly reduce the lubricant sensitivity compared to other fillers/binders. Variation of DP of the inulin can be utilized to control the tablet dissolution rate and drug release rate. In addition, inulin has the further advantages of being odorless, having a pleasant oral taste, and being less hygroscopic compared with other binders.

Xylitol

Xylitol is a pentahydroxy sugar-alcohol with the formula (CHOH)₃(CH₂OH)₂. Xylitol is a non-cariogenic sweetener and an alternative to sucrose in different categories of consumer products (i.e., food, supplements, and medicine). Xylitol can be produced via chemical hydrogenation of xylose or biotechnological processes. The chemical process requires high energy and production cost. The bioconversion process of xylitol involves specific microbial strains fermentation to hydrolyze xylose. Xylitol also has a very low order of toxicity through various routes of administration. FDA approved xylitol as an additive for food (21 C.F.R. § 172.395) in 1986 and approved it safe for human use.

From a sensory viewpoint, xylitol has a sweetness level equal to sucrose with a slight cooling effect upon dissolution in the mouth. Further, xylitol contains a lower calorie count than sucrose and is absorbed rapidly in the human body. From a healthcare viewpoint, xylitol is a “tooth-friendly” sugar alcohol with many dental health benefits such as tooth rehardening, tooth remineralization, as well as prevention of otitis, ear and upper respiratory infections It also has certain level of antimicrobial activity, which, for instance, inhibits the growth of microorganisms responsible for tooth decay. Besides, it is also consumed for diabetics to assist in treatment of hyperglycemia. Furthermore, it can also be utilized as prebiotics to enhance the probiotics in human body. In processes of various products (e.g., foods, drugs, and supplements), xylitol can be used as a coating material due to its pleasant cooling effect in oral and nasal cavities, as well as a stabilizing agent to prevent protein denaturation in process. Thus, it is a multi-functional ingredient with the combination of sweetener, binder, and healthcare benefits.

Lubricants

Lubricants are the excipients that are utilized to reduce friction via interposing an intermediate layer between tablet constituents and the die wall during compression and ejection. Most of the commonly-used lubricants are hydrophobic materials such as minerals (e.g., talc) and fatty acids or their derivatives (e.g., magnesium stearate), presence of which might also result in a less cohesive and mechanically weaker tablet. Screening and selecting appropriate lubricants and their quantity is of critical importance to the high quality of producing chewable tablets. Various lubricants may be used in the chewable tablet of the present disclosure. Non-limiting examples of suitable lubricants include magnesium stearate, stearic acid, ascorbyl palmitate, coconut oil powder, or rice bran extract. The wide range of lubricants used and tested in the present disclosure, from conventional magnesium stearate to innovative coconut oil powder, suggests a good compatibility and flexibility of the current excipient base formula with various lubricant agents.

Active Ingredients

The chewable formula of the present disclosure comprises at least one active ingredient and the previously described excipient base formulas. Various active ingredients may be used in the present disclosure. Non-limiting examples of active ingredients include vitamin(s) such as vitamin A, C, D₃, E, B₁, B₂, Niacin, B₆, Folate, B₁₂ or any combinations thereof; fruit extract; mineral(s) such as magnesium, phosphorous, copper, sodium fluoride, iron or any combinations thereof; food ingredient(s) such as protein, lipid, carbohydrate or any combinations thereof; or pharmaceutical agent(s) such as analgesics, antacids or laxative. In addition, various probiotics (e.g., Bacillus coagulans) and plant enzymes can be embedded into the disclosed excipient base formula for product development as well. These active ingredients help curing and preventing multiple nutrient-deficiency diseases. For instance, cobalamin, vitamin B₁₂, in the category of water-soluble vitamins, plays a critical role in the regular functions of brains and nervous system. (see, e.g., Reynolds, Vitamin B12, folic acid, and the nerve system, Lancet Neurol., 2006, 5(11), 949-960). B₁₂ deficiency might cause pernicious anemia in infant and elder populations. Sufficient amount of B₁₂ supplements can effectively prevent the occurrence of such nutrient-deficiency diseases, and enhance the brain health of human body.

Flavor Ingredients

Flavor ingredient is one sort of raw material substances which provide the senses of taste and smell to the products that utilized them. The flavor ingredients can be natural or artificial depending on their resources and processes. Natural flavor ingredients impart flavors that derive from natural substances (e.g., fruits, vegetables, poultry, etc.). On the contrary, artificial flavor ingredients are produced with flavors that do not derive from the natural substances. They are often chemical mixtures that mimic a natural flavor in taste or odor. These chemical compounds contribute to the specific taste or odor. For instance, limonene gives an orange juice odor; while ester-involved flavor compounds are sweet or fruity Once flavor is released in the mouth, the basic tastes (e.g., sweet, sour, bitter, salty and umami) or their combination will be recognized together with the specific sensations (e.g., fruity taste). (see, e.g., Bermúdez-Rattoni, Molecular mechanisms of taste-recognition memory, Nature Rev. Neurosci., 2004, 5, 209-217).

Flavor ingredients can be processed into liquid, powder, or solid crystals with different natural or chemical carriers. The carriers can be maltodextrin, modified starch, propylene glycol, or mixtures thereof. Different carriers and processes affect the physical and chemical properties of final flavor ingredients. The carriers of flavor ingredients can be corn syrup solid, maltodextrin, gum arabic and other vegetable gum(s), or the mixtures thereof. The carriers contribute to the improvement of finished flavor ingredients in various aspects. The carrier for flavor ingredient process can effectively improve dryer yield, increase the glass transition temperature of the powder, and enhance storage stability. For example, when orange juice concentrate powder is spray-dried with maltodextrin as a drying agent and dehumidified air as drying medium, the moisture content, hygroscopicity, and degree of caking of the processed powder decrease. The popular categories of flavor ingredients on the market include berry flavors (e.g., strawberry flavor), fruit flavors, vanilla flavors, coffee flavors, etc. Flavor ingredients can be re-mixed to form new flavor ingredients to have the complimentary effect of flavor notes. Flavor ingredients are usually used together with citric acid, sugar, and other ingredients (e.g., masking agent) to boost the flavor release and sensation, or diminish the unpleasant aftertaste. In some embodiments of the present disclosure, natural orange flavor and cherry flavor are used in chewable tablet formulas. These two flavor ingredients represent common flavors used for supplement products, especially chewable tablets herein.

Other Accessory Ingredients

Other accessory ingredients might be used for chewable tablet formulation, including but not limited to: dicalcium phosphate, calcium sulfate, clay, sodium lauryl sulfate (SLS), sodium benzoate, gums (e.g., xanthan gum, guar gum, and carrageenan), or chelating agent (e.g., EDTA). These additional ingredients have different functions depending on the specific needs of formulation. For instance, dicalcium phosphate serves as not only the calcium resource but also tableting agent in pharmaceutical preparation. Calcium sulfate is a desiccant for chewable tablet formula. Sodium lauryl sulfate is mainly used as detergent, surfactant, and emulsifier for chewable tablets. Sodium benzoate is a commonly used preservative for acidic foods (e.g., salad dressings, soda, fruit juice, etc.), medicines, and cosmetics. Sodium benzoate is also used to treat hyperammonemia. Gums such as xanthan gum are common thickening and stabilizing agents for various foods, pharmaceutical, and supplement applications. Different gums have distinct physical- and chemical-properties. For example, carrageenan can strongly interact with calcium or other positive-charged salts to form solid gel at high concentrations [see, e.g., U.S. Pat. No. 3,956,173, incorporated herein by reference]. Ethylenediamine tetraacetic acid (EDTA) is a metal ion-chelating agent to prevent heavy metal poisoning and softening the medium.

Process of Chewable Tablets

In certain embodiments, the chewable formula is composed of at least one active ingredient, from about 15% to about 40% by weight of xylitol, from about 20% to about 40% by weight of inulin, from about 1% to about 3% by weight of flowing agent (e.g., silica), and from about 1% to about 4% by weight lubricant(s), based on the total weight of the chewable formula. In other embodiments, from about 3% to about 6% by weight of flavor can also added.

Further embodiments of the present application relate to a process for preparing chewable tablet from the aforementioned chewable formulas.

The process of chewable tablet production can be either direct compaction or with the aid of extra steps (e.g., dry/wet granulation). The granulation process is utilized to render powder particles adhere to form larger particles or granules, which might improve the quality of tableting depending on formulas. For instance, the wet granulation process typically involves wet massing the solid ingredients of chewable formula with a liquid (e.g., water, ethanol, or isopropyl alcohol) to form wet aggregates. Then, the liquid is removed from the wet aggregates to form dry aggregates, followed by milling the dried aggregates to an appropriate size. Overwetting of granules in the wet granulation process can produce harder granules. Chewable tablets made from such wet granulations often have a gritty texture when chewed. This grittiness can be reduced by using a direct compaction manufacturing process and appropriate excipients with suitable physical-chemical properties which eliminates the wet massing and subsequent drying step.

In one particular embodiment, the chewable tablet of present disclosure is produced by a direct compaction process. In some embodiments, the method may include preparing the excipient base formula; blending the excipient base formula with at least one active ingredient at an appropriate weight ratio to obtain a chewable formula. The chewable formula is then compressed into a tablet of appropriate size and shape. In some embodiments, all the desired ingredients may be mixed to assure homogeneity in an appropriate blender, followed by compressing the homogeneous mixture into a tablet of appropriate size and shape. The prior-tableting process (e.g., mixing and blending) can be tailored according to the demand of chewable tablet formulas.

Tablet compression tooling (e.g., punches and dies) with various punch sizes and shapes may be used in the direct compaction. Examples of punch size may include, but are not limit to, a 7/16″ round concave shape, a ⅝″ round flat shape, a ⅞″ round flat shape, or a ⅞″ round concave dimple shape.

Physical Characterization

Certain physical characteristics of chewable formula are required to achieve the good quality of chewable tablet, including flowing property, stickiness, and tabletability. Good flowing property of chewable formula ensures a smooth powder flow of chewable formula during tableting and a controllable tablet weight with small deviation. Sticking causes tableting problem since the powder of chewable formula might be sticking to the toolings (punches and dies), and the situation becomes worsen as tableting goes on. Raw materials (e.g., lubricants) with hydrophobic features commonly have anti-sticking performance. Selecting appropriate lubricants can effectively reduce the occurrence of sticking issue during tableting. Tabletability is also critical for producing chewable tablets of high quality. Tablets with too low tabletability can result in the formation of soft tablets that easily get broken during transportation and handling. Tablets with too high hardness might harm teeth through mouth-chewing. Manipulation of material crystals through appropriate processes (e.g., surface modification) helps improve the material's poor tabletability (e.g., hydrophobic ibuprofen) [see, e.g., Overcoming poor tabletability of pharmaceutical crystals by surface modification, Pharma. Res., 2011, 28(12), 3248-3255]. Chewable tablets without the issues of capping, chipping, lamination and out-of-spec (OOS) of hardness and % friability parameters are commonly considered as chewable tablets with good tabletability.

Two critical criteria in the quality of a tablet are hardness and friability. The resistance of the tablet to chipping, abrasion, or breakage under conditions of storage, transportation and handling before usage depends on its hardness. Hardness is measured by determining lateral breaking strength exerted on a single tablet at the moment of rupture. Hardness is expressed in kilo pounds (kp) or Strong Cobb Units (S.C.U.), wherein 1 kp equals to 1.4 S.C.U. Acceptable hardness depends on the desired feeling or texture, the expected end use, and packaging conditions of the tablet. In most contexts, tablet hardness must be greater than 10 S.C.U. to be commercially useful. The hardness of regular tablets can go up to 35 kp or more. For chewable tablets, the hardness is commonly controlled within 25 kp for the convenience of mouth-chewing. The tablet hardness also depends on the tablets' punch size. Tablets with larger size require set-up of higher hardness, in other words, higher kp values.

Friability is measured under standardized conditions by weighing out a certain number of tablets (generally 20 or more), and placing them in a rotating plexiglass drum in which they are lifted during replicate revolutions by a radial louver and then dropped through the diameter of the drum. After replicate revolutions, the tablets are reweighed and the percentage of powder “rubbed off” or broken pieces is calculated. Friability in the range of about 0% to 3% is considered acceptable for most drug and food tablet contexts. Friability which approaches 0% is particularly preferred.

In some embodiments, the chewable tablet is formed from the chewable formula that comprises at least one active ingredient, from about 15% to about 40% by weight of xylitol, from about 20% to about 40% by weight of inulin, from about 1% to about 3% by weight of flowing agent (e.g., silica), and from about 1% to about 4% by weight lubricant, based on the total weight of the chewable tablet. Particular embodiments can include from about 3% to about 6% by weight of flavor.

In further embodiments, the fruit extract chewable tablet is formed from the fruit extract chewable formula that comprises from about 30% to 40% of fruit extract; from about 10% to about 40% by weight of xylitol; from about 20% to about 40% by weight of inulin; from about 1% to about 3% by weight of flowing agent; and from about 1% to about 4% by weight lubricant, based on total weight of the chewable tablet. Particular embodiments can include from about 3% to about 6% by weight of flavor.

The excipient base formula of present disclosure has several advantages over the conventional excipient base formula of the prior art. In addition to alleviate the sticking and tabletability issues during tablet manufacture, the excipient base formula of present disclosure allows for a one-step, controllable direct compaction process with simple blending. It shows excellent performance in a 12-week accelerated stability test of chewable tablets developed based on this excipient base formula, indicating a sustainable shelf life of chewable products. It has very little or no taste, thus facilitating the incorporation of active ingredients and flavors. Even better, the excipient base formula enhances the flavor release and overall sensation of chewable products. Moreover, it meets the demand of clean label for marketing in the United States.

### EXAMPLES

### Example 1—Materials

Granulated xylitol with a very sweet cool taste was obtained from Danisco Sweetener (DuPont Nutrition & Health, Wilmington, Del.). Inulin agave was obtained from Tic Gum (Belcamp, Md.). Syloid 244 FP silica was obtained from GRACE (Columbia, Md.). Various lubricants were used, such as stearic acid from AIC, ascorbyl palmitate from Pharmline (Florida, N.Y.), coconut oil powder from Stauber (Fullerton, Calif.), magnesium stearate from Anhui Sunhere Pharmaceutical Excipients Co., Ltd. (Huinan, China), or Rice bran extract from Ribus, Inc. (St. Louis, Mo.).

Table 1 shows the physical properties of each ingredient used in the excipient base formula, specifically describing sensory qualities of each ingredient. The clean sensory attributes of these ingredients help building-up chewable formulas of high quality.

TABLE 1
Physical Properties of Ingredients used in Excipient Base Formula

| | Physical Properties | | | |
|---|---|---|---|---|
| | Physical | | | Molecular |
| Ingredient | form | Taste | Odor | Weight |
| Xylitol | Granules | Very sweet | Odorless | 152 |
| Inulin | Fine powder | Slightly sweet | Odorless | 5000 |
| Silica | Fluffy powder | Bland | Odorless | 60 |
| Stearic acid | Fine powder | Bland | Odorless | 284 |
| Coconut | Fine powder | Bland | Odorless | 210 |
| oil powder | | | | |

### Example 2—Excipient Base Formula

The excipient base formula was studied for the development and production of fruit extract chewable tablets, including Formula A, Formula B, and Formula C; and magnesium chewable tablets of Formula D, Formula E, and Formula F. Each ingredient in excipient base formula has a percentage range for developing specific chewable formulas, which gives a certain degree of formulation flexibility. The components for excipient base formula were as listed in Table 2 by % weight range based on total weight of the excipient base formula. The fruit extract chewable tablets (e.g., Formula A, Formula B, and Formula C) and the magnesium chewable tablets (e.g., Formula D, Formula E, and Formula F) are developed using the excipient base formula. The other accessory ingredients can be acids, gums, clay, surfactant (e.g., sodium lauryl sulfate), antimicrobial agents (e.g., sodium benzoate), and chelating agent (e.g., EDTA), which render the entire formula to 100%.

TABLE 2
Excipient Base Formula

| | Broad Range | Narrower Range |
|---|---|---|
| Component | (% weight) | (% weight) |
| Xylitol | 15%-55% | 15%-40% |
| Inulin | 20%-45% | 20%-40% |
| Silica | 1%-4% | 1%-3% |
| Lubricant | 1%-4% | 1%-4% |
| Flavor | 3%-10% | 3%-6% |
| Accessory Ingredients | — | — |

### Example 3—Fruit Extract Chewable

The fruit extract chewable formulas (Formula A, Formula B, and Formula C) were prepared by blending the corresponding excipient base formula with an active ingredient (fruit extract). Thereafter, fruit extract chewable tablets were produced by subjecting the fruit extract chewable formula to a direct compaction via Stokes DD2 31 Station Tablet Press using ⅝″ round flat punch size. The components of Formula A, Formula B, and Formula C were as listed in Table 3, % weight based on total weight of the chewable formula.

TABLE 3
Chewable Formula for the Production of Fruit Chewable Formula
(by % weight based on total weight of the chewable formula)

| Ingredient | Formula A | Formula B | Formula C |
|---|---|---|---|
| Fruit Extract | 36.7% | 36.0% | 33.5% |
| Xylitol | 14.7% | 28.8% | 35.6% |
| Inulin | 36.7% | 21.6% | 23.5% |
| lavor | 3.0% | 3.2% | 3.4% |
| Citric acid | 4.4% | 4.0% | 1.0% |
| Silica | 3.0% | 2.5% | 1.3% |
| Stevia | — | — | 0.4% |
| Lubricant | 1.5% | 4.0% | 1.3% |
| Total | 100.0% | 100.0% | 100.0% |

Formula A was used to produce cherry fruit extract chewable tablets. Formula A contained 36.7% by weight of inulin, which led to a high tablet hardness (up to 20 kp) without friability issues (<2%) when compaction occurred. The citrus cherry taste quickly boosted the flavor release in the mouth, and offered a good quantity of anthocyanin for human nutrition. It has been reported that anthocyanins have antioxidant properties in vitro [see, e.g., Einbond et al., Anthocyanin antioxidants from edible fruits, Food Chem., 2004, 84(1), 23-28]. Clinical studies also showed that supplementing anthocyanin possibly plays a role in the prevention or treatment of chronic inflammatory diseases by inhibiting NF-κB transactivation and decreasing plasma concentrations of various signaling proteins secreted by cells [see, e.g., Karlsen et al., Anthocyanins inhibits nuclear factor κB activation in monocytes and reduce plasma concentrations of pro-inflammatory mediators in healthy adults, J. Nutr., 2007, 137(8), 1951-1954].

The chewable tablets of Formula A were subjected to the accelerated stability test. The length of the test and the storage condition were sufficient to cover typical storage, shipment and subsequent use. The chewable tablets of Formula A were stored in 250-ml polyethylene terephthalate (PET) bottles during 12-week accelerated stability test. The condition of 40° C.±2° C. at 75%±5% RH and the period of three months were adopted in the accelerated stability test for this disclosure according to ICH Q1A.

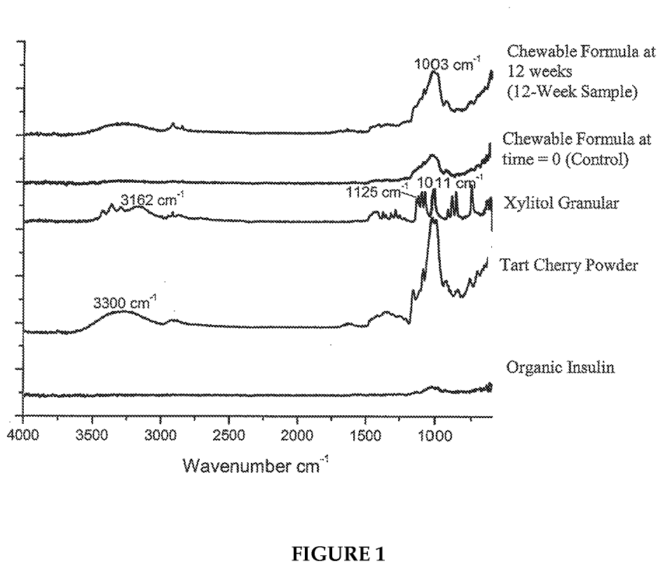

FIG. 1 shows the Attenuated Total Reflectance (ATR)—Fourier Transform Infrared (FTIR) spectra of the control chewable tablets of Formula A (i.e., at time=0), the 12-week chewable tablets of Formula A (i.e., at time=12 weeks under the accelerated stability test), granular xylitol, tart cherry, and organic inulin. The ATR-FTIR spectra for detecting functional groups' motions were collected to verify the structure alternation. Table 4 shows the feature bands' assignments from ATR-FTIR spectra.

TABLE 4
Feature bands' assignments from ATR - FTIR spectra

| Wavenumber, cm⁻¹ | Assignment |
|---|---|
| 1003 | C—O stretching |
| 1011-1125 | C—C stretching, C—O stretching |
| 3162 | C—H stretching |
| 3300 | O—H stretching (Hydrogen bonding) |

Prior to measurement, the powder samples were all compressed into small solid pellets for the convenience of measurements. The solid chewable tablets were directly subjected to the ATR-FTIR measurement without additional treatment. ATR-FTIR spectra of the chewable tablets and powder samples were collected at ambient temperature with the aid of a Thermo Nicolet 670 FT-IR Spectrometer (Thermo Electron Corp, Madison, Wis.). The spectra were collected over 512 scans at 4 cm⁻¹ resolution using a smart miracle accessory. The representative samples were carefully selected and were directly pressed onto Ge crystal for the measurement.

FTIR spectroscopy is a technique to collect infrared spectra of absorption or emission of a solid, liquid and gas. FTIR spectrometer with an attenuated total reflectance (ATR) attachment provides high-resolution data covering a wide spectral range or band range. This technique observes the characteristic molecular behaviors under the scanning of beamline with a broad range of frequencies, which potentially suggests specific bond before/after synthesis or degradation, molecular interaction (i.e., hydrogen bonding), and structure prediction. FIG. 1 displays the FTIR-ATR spectra of the control chewable tablets (i.e., at time=0), the 12-week chewable tablets (i.e., at time=12 weeks under the accelerated stability test), granular xylitol, tart cherry, and inulin. The characteristic bands for the IR curves of all the samples are summarized in the Table 4.

As shown in FIG. 1, the major IR band is located at 1003 cm′, which is contributed by —C—O stretching on the anthocyanin molecules from tart cherry. The IR absorption from tart cherry is dominant among IR signals' contribution from other ingredients such as inulin, and xylitol. It is clear that the feature bands from granular xylitol (1011-1125 cm⁻¹), indicating —C—C and —C—O stretching, do not appear in the IR curves of chewable tablet samples. The band at 3300 cm⁻¹ is due to —O—H stretching, suggesting the hydrogen bonding among molecules. Comparing the control chewable tablet sample and the 12-week chewable tablet sample, there is no major IR band changes (e.g., no appearance of new IR bands, or disappearance of original IR bands). A broad band at 3300 cm⁻¹ from —O—H stretching is most likely due to the fact that the increase of water activity inside of chewable tablet after 12-week accelerated stability test leads to the dynamic hydrogen boding. Nonetheless, no major chemical reaction or degradation is found between the control sample and the 12-week sample based on the IR measurement.

The chewable tablets of Formula A were also subjected to the organoleptic test, KP, and friability % measurement. Table 5 shows the organoleptic test, KP, and friability % measurement of the chewable tablets of Formula A at time=0 (i.e., “Formula A—Control”), in comparison to the chewable tablets of Formula A after 12 weeks of the accelerated stability test (i.e., “Formula A—12 Weeks”).

TABLE 5
Organoleptic Test, KP, and Friability % Measurement

| Sample | Organoleptic Test | KP | Friability % |
|---|---|---|---|
| Formula A - Control | 85% Consistent | 16 | 99.8 |
| Formula A - 12 Weeks | | 23 | 99 |

The panel-based sensory taste test (organoleptic test) was performed by a 15-person group of professional individuals with the age ranging from 20 to 60. Each individual was treated to taste “Formula A—Control” and “Formula A—12 weeks” samples under fasting condition prior to giving their taste result. Cool water or salty crack was taken between intakes of different chewable tablet samples to eliminate the residue taste from the previously-tasted samples. After organoleptic test, each panelist gave the descriptive results stating whether the overall taste of “Formula A—Control” and “Formula A—12 weeks” samples was consistent with each other.

Table 5 displays the comparison of sensory and physical attributes between “Formula A—Control” and “Formula A—12 weeks” samples. The friability % of “Formula A—Control” after 12-week accelerated stability test did not change (<1%), and maintained within 1%. The tablet hardness of Formula A increased from 16 kp to 23 kp after 12-week accelerated stability test, which might be due to the increase of water activity in chewable tablet in the high relative humidity environment during the stability test. It is in agreement with the observation of a broad band at 3300 cm⁻¹ in IR curve of “Formula A—12 weeks” sample. The physical test results (both tablet hardness and friability %) of “Formula A—12 weeks” sample suggest a robust chewable tablet during bottling, transportation, and storage potentially. Regarding the sensory perspective of “Formula A—Control” sample and “Formula A—12 weeks” sample, most of the panelists who participated in the organoleptic test (85%) considered the overall taste of both samples were consistent.

Formula B was used to produce other cherry fruit extract chewable tablets. Formula B contained a higher xylitol content and lower inulin content, compared to Formula A. As such, Formula B had a sweeter sensation profile and lower hardness (up to 14 kp), compared to Formula A. Although tablet hardness of Formula B is lower than Formula A, higher amount of xylitol leads to a sweeter sensory profile of Formula B. Thus, tuning the contents of xylitol and inulin in the excipient base formula resulted in different chewing hardness and organoleptic sensation (e.g., sweetness).

Formula C was used to produce camu berry fruit extract chewable tablets. The usage of more sweeteners (e.g., xylitol, and stevia, a high potency natural sweetener) was applied to further enhance the sweet sensation and decrease the citrus sensation, which reflects a formula flexibility of the Excipient Base Formula in this disclosure.

### Example 2. Magnesium Chewable

Further, the chewable formulas of Formula D, Formula E, and Formula F were used for the production of magnesium chewable tablets. The components for each magnesium chewable tablets were as listed in Table 6. The chewable formulas of Formula D, Formula E, and Formula F were also developed based on the excipient base formula in this disclosure. As shown in Table 6, Formula D, Formula E, and Formula F were similar to each other, and the only difference was the type of binder used in the formula (i.e., inulin in Formula D, cellulose in Formula E, and sorbitol in Formula F).

TABLE 6
Chewable Formulas for the production of Magnesium Chewable Tablets
(by % weight based on total weight of the chewable formula)

| Formula D | | Formula E | | Formula F | |
|---|---|---|---|---|---|
| | Per- | | Per- | | Per- |
| Ingredient | centage | Ingredient | centage | Ingredient | centage |
| Magne- | 24.0% | Magne- | 24.0% | Magne- | 24.0% |
| sium salt | | sium salt | | sium salt | |
| Xylitol | 29.6% | Xylitol | 29.6% | Xylitol | — |
| Inulin | 29.6% | Cellulose | 29.6% | Sorbitol | 59.2% |
| Flavor | 5.1% | Flavor | 5.1% | Flavor | 5.1% |
| Citric acid | 7.1% | Citric acid | 7.1% | Citric acid | 7.1% |
| Silica | 1.6% | Silica | 1.6% | Silica | 1.6% |
| Stevia | 0.5% | Stevia | 0.5% | Stevia | 0.5% |
| Lubricant | 2.5% | Lubricant | 2.5% | Lubricant | 2.5% |
| Total | 100.0% | | 100.0% | | 100.0% |

The magnesium chewable tablets of Formula D, Formula E and Formula F were subjected to the panel-based sensory taste test. The sensory results were used to provide a semi-quantitative evaluation and comparison among chewable tablet samples from sensory perspective. The test was performed by a 15-person group of professional individuals with the age ranging from 20 to 60. Each individual was treated to taste Sample D (i.e., the chewable tablet of Formula D), Sample E (i.e., the chewable tablet of Formula E), and Sample F (i.e., the chewable tablet of Formula F) under fasting condition prior to giving their taste result. Cool water or salty crack was taken between intakes of different chewable tablet samples to eliminate the residue taste from the previously-tasted samples. The taste test results were based on the score assignments for the five organoleptic attributes, including sweetness, acidity, flavor release, texture, and aftertaste. The basic taste of magnesium chewable tablet should be an orange taste with a combined profile of sour/sweet-oriented sensation. A little bit sourer taste helps stimulating the human saliva and accelerating the experience of organoleptic test. The overall performance index (OPI) was used to provide a semi-quantitative evaluation of each chewable tablet. The calculation of OPI took different weights for different sensory attributes. For example: sweetness and acidity were the two most important sensory attributes, and their weights in calculating OPI were higher than other sensory attributes such as texture, flavor release, and aftertaste. The equation for calculating OPI is illustrated by Eq. 1, the overall performance index equation as:

Sweetness*0.25+Acidity*0.3+Flavor Release*0.15+Texture*0.2+Aftertaste*0.1 Eq.

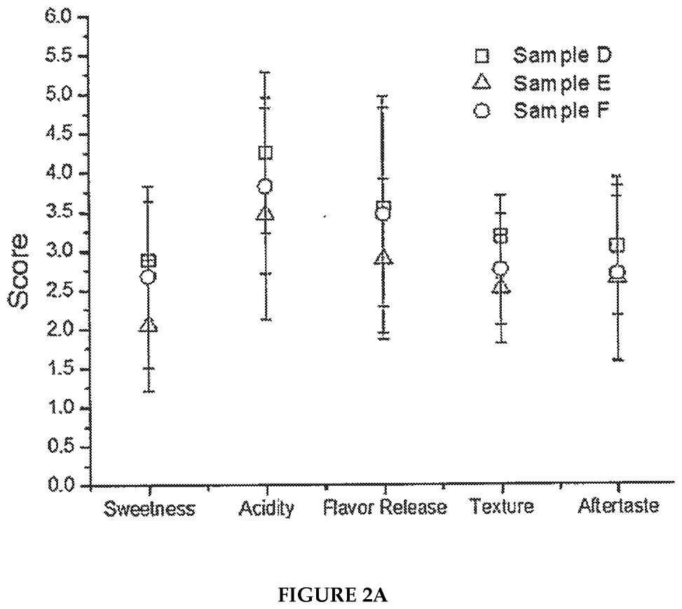

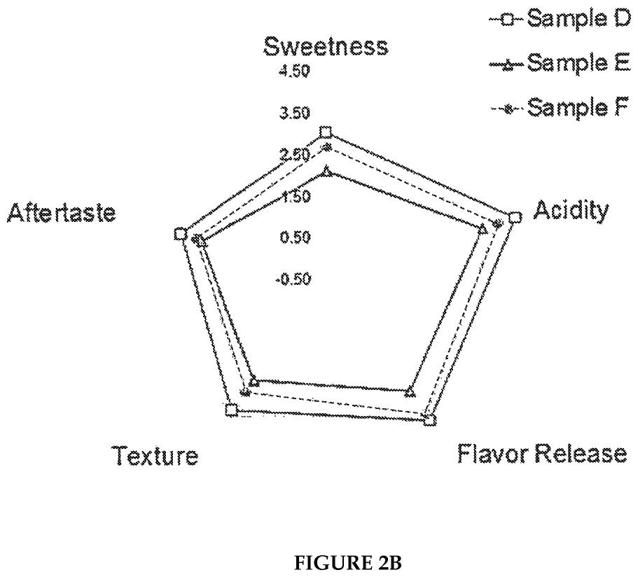

Panel-based sensory taste test or organoleptic test was utilized to evaluate the edible magnesium chewable tablet based on the sensory attributes' performance (e.g., sweetness, acidity, and texture). The sensory results were used to provide a semi-quantitative evaluation and comparison among chewable tablet samples from sensory perspective. FIG. 2 displays the results of sensory taste test on chewable tablet samples of Formulas D, E, and F. FIG. 2A shows the sensory attribute scores (e.g., sweetness, acidity, and texture) of chewable tablet samples of Formulas D, E, and F. Among the three samples, the sample of Formula E had the lowest scores in all five sensory attributes; while the scores for the samples of Formula D and F were more similar to each other. The scores of sweetness, acidity, and texture for the sample of Formula D were higher than those for the sample of Formula F. In addition, sensory spider plots for the chewable tablet samples of Formulas D, E, and F were drawed for a more direct observation in FIG. 2B. It is clear that the chewable tablet sample of Formula D had the largest plot contour among the three samples, suggesting an leading overall sensory performance. The difference between the chewable tablet samples of Formulas D, E, and F was the use of different binders/sweeteners, which involves inulin in the sample of Formula D, cellulose in the sample of Formula E, and sorbitol in the sample of Formula F. The sweetness level of these three excipients is sorbitol>inulin>cellulose. Cellulose is a traditional binder for swallowing tablet direct/indirect compaction. In sensory test herein, cellulose in the sample of Formula E served as a flavor and texture masking agent to retard the flavor release and delay the overall mouth sensation. The gummy chewing texture from cellulose lowered the texture feeling of the sample of Formula E. Sorbitol in the sample of Formula F is the sweetest binder/sweetener of the three, which, however, did not aid the sample of Formula F to compete the most popular among the three samples. On the contrary, inulin in sample of Formula D helped the formula target the highest scores in all five sensory attributes, indicating a unique sensory contribution of inulin in chewing supplements. The largest overall contour of spider plot of the sample of Formula D with inulin illustrated a well-round, balanced sensory contribution by inulin towards chewable formula. This result reflected a sensory advantage of inulin for chewing supplements compared with other traditional binders and sweeteners (e.g., sorbitol and cellulose).

While the present invention has been described herein with respect to certain preferred embodiments, those of ordinary skill in the art will recognize and appreciate that it is not so limited. Rather, many additions, deletions, and modifications to the preferred embodiments may be made without departing from the scope of the invention as hereinafter claimed. In addition, features from one embodiment may be combined with features of another embodiment while still being encompassed within the scope of the invention as contemplated by the inventors.

We claim:

1. An excipient base formula for chewable tablet, comprising:
from about 15% to about 55% by weight of xylitol;
from about 20% to about 45% by weight of inulin;
from about 1% to about 4% by weight of flowing agent; and
from about 1% to about 4% by weight of lubricant, based on the total weight of the excipient base formula.

2. The excipient base formula of claim 1, comprising:
from about 15% to about 40% by weight of xylitol;
from about 20% to about 40% by weight of inulin;
from about 1% to about 3% by weight of flowing agent; and
from about 1% to about 4% by weight lubricant, based on the total weight of the excipient base formula.

3. The excipient base formula of claim 1, further comprising from about 3% to about 10% by weight of flavoring agent based on the total weight of the excipient base formula.

4. The excipient base formula of claim 1, wherein the flowing agent comprises silica.

5. The excipient base formula of claim 1, wherein the lubricant comprises a material selected from the group consisting of magnesium stearate, stearic acid, ascorbyl palmitate, coconut oil powder, and rice bran extract.

6. A chewable formula comprising at least one active ingredient and the excipient base formula of claim 1, wherein the inulin is not considered as the active ingredient.

7. The chewable formula of claim 6, wherein the chewable formula is directly compressible into a chewable tablet.

8. A chewable tablet produced from the chewable formula of claim 6.

9. The chewable tablet of claim 8, wherein the at least one active ingredient comprises a substance selected from the group consisting of vitamin, fruit extract, mineral, food ingredient, and pharmaceutical agent.

10. The chewable tablet of claim 9, wherein the vitamin comprises vitamin A, C, D₃, E, B₁, B₂, Niacin, B₆, Folate, B₁₂, or any combinations thereof.

11. The chewable tablet of claim 9, wherein the mineral comprises magnesium, phosphorous, copper, sodium fluoride, iron, or any combinations thereof.

12. The chewable tablet of claim 9, wherein the food ingredient comprises protein, enzyme, probiotics, lipid, carbohydrate, or any combinations thereof.

13. The chewable tablet of claim 9, wherein the pharmaceutical agent comprises analgesics, antacids or laxative.

14. A chewable formula, comprising:
at least one active ingredient
from about 15% to about 40% by weight of xylitol;
from about 20% to about 40% by weight of inulin;
from about 1% to about 3% by weight of flowing agent; and
from about 1% to about 4% by weight of lubricant based on the total weight of the chewable formula, wherein the inulin is not considered as the active ingredient.

15. The chewable formula of claim 14, wherein the chewable formula is directly compressible into a chewable tablet.

16. A chewable tablet produced from the chewable formula of claim 14.

17. A fruit extract chewable formula, comprising:
from about 30% to 40% of fruit extract;
from about 10% to about 40% by weight of xylitol;
from about 20% to about 40% by weight of inulin;
from about 1% to about 3% by weight of flowing agent; and
from about 1% to about 4% by weight lubricant based on total weight of the chewable tablet, wherein the inulin is not considered as the active ingredient.

18. A method of producing a chewable tablet, comprising:
preparing the excipient base formula of claim 1;
mixing the excipient base formula of claim 1 with at least one active ingredient to obtain a chewable formula; and compressing the chewable formula into a tablet form.

19. A method of producing a chewable tablet, comprising:
preparing the chewable formula of claim 14; and
compressing the chewable formula into a tablet form.

20. A method of producing fruit extract chewable tablet, comprising:
preparing the fruit extract chewable formula of claim 17; and
compressing the fruit extract chewable formula into a tablet form.
